(19) United States
(12) Patent Application Publication
(10) Pub. No.: US 2002/0165122 A1
(43) Pub. Date: Nov. 7, 2002

(54) Method and compositions for the treatment or amelioration of female sexual dysfunction

(76) Inventors: Jeremy P. W. Heaton, Kingston (CA); Michael A. Adams, Kingston (CA)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 10/136,387

(22) Filed: May 2, 2002

Related U.S. Application Data
(63) Continuation of application No. 09/102,987, filed on Jun. 22, 1998.
Application No. 09/102,987 is a continuation-in-part of application No. 08/546,498, filed on Oct. 20, 1995, now Pat. No. 5,770,606.
Application No. 08/546,498 is a continuation-in-part of application No. 08/231,250, filed on Apr. 22, 1994, now abandoned.

(51) Int. Cl. A61K 31/00

(52) U.S. Cl. 514/1

(57) ABSTRACT

The present invention provide a method of treating sexual dysfunction in a female, including the vasculogenic symptoms of delayed vaginal engorgement, diminished vaginal lubrication, pain or discomfort with intercourse (dyspareunia), diminished vaginal sensation, diminished vaginal orgasm, diminished clitoral sensation or diminished clitoral orgasm, or of combating vaginal pain by stimulating peripheral pelvic nerve release of nitric oxide (NO). The method comprises administering to a female in need of such treatment a therapeutically effective amount of a compound which acts on a mid-brain pathway to increase blood flow to the ilio-hypogastric-pudendal artery bed and stimulate the release of nitric oxide (NO) from peripheral NANC nerve cells. The preferred compound for the method of this invention is apomorphine or one of its pharmaceutically acceptable salts, esters, or pro-drugs.

### CROSS-REFERENCE TO RELATED APPLICATIONS

This application is a continuation-in-part of co-pending application, Ser. No. 08/546,498 filed Oct. 20, 1995, now U.S. Pat. No. 5,770,606 which, in turn, is a continuation-in-part of application Ser. No. 08/231,250 filed on Apr. 22, 1994, now abandoned.

### FIELD OF THE INVENTION

The present invention relates to pharmaceutical compositions and to a medical method of treatment. More particularly, the present invention concerns pharmaceutical compositions containing a compound which acts upon a mid-brain neural pathway to increase blood flow in the ilio-hypogastric-pudendal arterial bed to treat or ameliorate vasculogenic sexual dysfunction and to stimulate peripheral release of nitric oxide (NO) to combat vaginal pain in female mammals.

### BACKGROUND OF THE INVENTION

Sexual response in mammals is mediated by a balanced interplay between the sympathetic and parasympathetic nervous systems. Vasocongestion, or erectile tumescence in both the male and female, is largely mediated by parasympathetic (cholinergic) outflow, whereas orgasm is predominantly sympathetic (adrenergic).

Sexuality in human females encompasses multiple components including physiological, psychological, social and emotional factors. However, the first phase of the female sexual response is mediated by a combination of vasocongestive and neuromuscular events which include increased clitoral length and diameter, as well as increased vaginal lubrication, wall engorgement and increased luminal diameter.

The clitoris is the homologue of the penis, arising from the embryological genital tubercle. As a result, the two organs have similar structural and arousal response mechanisms. The clitoris consists of a cylindrical, erectile organ composed of three parts: the outermost glans or head, the middle corpus or body, and the innermost crura. The body of the clitoris consists of paired corpora cavernosa of about 2.5 cm in length and lacks a corpus spongiosum. During sexual arousal, blood flow to the corpora cavernosa of the clitoris cause their enlargement and tumescence.

The clitoris plays a major role during sexual activity in that it induces local autonomic and somatic reflexes causing vaginal vasocongestion, engorgement, and subsequent transduction, lubricating the introital canal making the sexual act easier, more comfortable, and more pleasurable.

Vaginal wall engorgement enables a process of plasma transduction to occur, allowing a flow through the epithelium and onto the vaginal surface. Plasma transduction results from the rising pressure in the vaginal capillary bed during the sexual arousal state. In addition, there is an increase in vaginal length and luminal diameter, especially in the distal ⅔ of the vaginal canal.

The vaginal canal is lubricated primarily from a transudate originating from the subepithelial vascular bed passively transported through the interepithelial spaces sometimes referred to as intercellular channels. Additional moistening during intercourse comes from secretion of the paired greater vestibular or Bartholin's glands.

These events depend upon sufficient blood flow to these organs during sexual arousal, and a physiologic disorder which impairs this blood flow, resulting in female vasculogenic sexual dysfunction, can ultimately lead to or exacerbate a pre-existing psychological condition.

The arterial supply to the vagina is derived from an extensive network of branching vessels surrounding it from all sides. The anterior branch of the internal iliac artery continually bifurcates as it descends through the pelvis with a series of the newly generated vessel, each supplying the vagina to some degree. After giving off an obturator artery branch, the umbilical and the middle rectal arteries diverge off to supply a superior and inferior vesical artery, respectively. Between the umbilical and the mid-rectal branches there is generation of a uterine artery which further bifurcates to give the vaginal artery. The internal pudendal and accessory pudendal artery also sends a branch. Finally the common clitoral artery sends a branch to the vaginal muscularis.

The main arterial supply to the clitoris from the ilio-hypogastric-pudendal arterial bed. The internal pudendal artery is the last anterior branch of the internal iliac artery. Distally, the internal pudendal artery traverses Alcock's canal, a position of the obturator fascia and lies on the inner side in supposition to the ischio-pubic ramis. In this latter location, the artery is susceptible to blunt perineal trauma. The internal pudendal artery terminates as it supples the inferior rectal and perineal artery, which supplies the labia. The common clitoral artery continues to the clitoris. This artery bifurcates into a dorsal clitoral artery and a cavernosal clitoral artery.

Based upon animal research, it has been found that central nervous system areas primarily implicated in sexual arousal include the medial pre-optic, anterior hypothalamic region and related limbic-hippocampal structures of the brain.

Female sexual dysfunction which has its origin in abnormal arterial circulation into the vagina or clitoris during sexual stimulation may be considered a disorder of arousal. This vasculogenic female sexual dysfunction may include such clinical symptoms as delayed vaginal engorgement, diminished vaginal lubrication, pain or discomfort with intercourse (dyspareunia), diminished vaginal sensation, diminished vaginal orgasm, diminished clitoral sensation or diminished clitoral orgasm.

Moreover, traumatic injury to the ilio-hypogastric-pudendal arterial bed from pelvic fractures or blunt perineal trauma may also result in diminished vaginal/clitoral blood flow following sexual stimulation and fall into the vasculogenic dysfunction category.

Vaginal pain may derive from a general vaginal hyperalgesia or sensitivity to stimulation of from the pain associated with coitus (dyspareunia) when there has been sufficient genital engorgement and lubrication.

Treatment of female sexual dysfunction is gradually evolving as more clinical and basic science studies are dedicated to the investigation of this medical problem. Female sexual complaints are not all psychological in pathophysiology, especially for those individuals who may have a component of vasculogenic dysfunction contributing to the overall female sexual complaint. Aside from hormone replacement therapy, medical management of female sexual dysfunction remains in the early phases of development. All non-hormonal medications listed below are undergoing safety and efficacy testing for the treatment of male erectile dysfunction and are only in the experimental stage for the treatment of female sexual dysfunction.

Estrogen replacement therapy is presently used in post-menopausal women (either spontaneous or surgical) for the treatment of hot flashes, prevention of osteoporosis, and diminishment of the risk of heart disease. Estrogen replacement results in improved clitoral sensitivity, increased libido and decreased pain/burning during intercourse. Local or topical estrogen application relieves symptoms of vaginal dryness, burning, urinary frequency and urgency. No clinical evidence exists thus far that the use of topical estrogen cream results in relief of sexual complaints other than local vaginal pain or vaginal dryness.

Methyl testosterone may be used in combination with estrogen in post-menopausal women for symptoms of inhibited desire, dyspareunia or lack of vaginal lubrication. Topical vaginal testosterone is used for treatment of vaginal lichen planus. These women, usually elderly, are noted to have clitoral enlargement, increased facial hair and increased sexual appetite. There are conflicting reports regarding the benefit of methyl testosterone for the treatment of inhibited desire and/or vaginismus in pre-menopausal women.

In men, topical application of prostaglandin E1 combined with a skin enhancer such as SEPA is presently demonstrating initial success in pilot Phase II clinical trials. Clinical studies are necessary to determine the safety and efficacy of this medication used as a topically-administered vaginal vasoactive agent in the treatment of vasculogenic female dysfunction. However, one study has demonstrated increased clitoral blood flow and clitoral erection following local prostaglandin E1 injection into clitoral corporal erectile tissues.

Sildenafil functions as a selective type 5 (i.e. c-GMP specific) phosphodiesterase inhibitor, and acts to decrease the metabolism of c-GMP, the second messenger in nitric oxide mediated male erectile response. An oral formulation of this medication has proven to be safe and effective in improving erectile duration and rigidity. In females, nitric oxide/NOS exists in human vaginal and clitoral tissue. Sildenafil may prove useful alone, or possibly in combination with other vasoactive agents for the treatment of vasculogenic female sexual dysfunction. Clinical studies evaluating the efficacy of this medication in women are needed.

Phentolamine is currently available as an oral preparation with rapid absorption and metabolism. Phentolamine's mechanism of action inducing vascular smooth muscle relaxation occurs via alpha-adrenergic blockade as well as by direct smooth muscle relaxation. Studies are currently in progress using this medication in women with female sexual dysfunction.

Despite these advances in the discovery of agents effective to treat female sexual dysfunction, there still exists a need for the discovery of additional compounds useful in the treatment of this condition.

### SUMMARY OF THE INVENTION

In one embodiment, the present invention provides a method of treating or ameliorating vasculogenic sexual dysfunction in female mammals by administering to a mammal in need of such treatment a therapeutically effective amount of a compound which acts upon mid-brain pathways to increase blood flow to the ilio-hypogastric-pudendal arterial bed.

In another embodiment, the present invention provides a method of combating vaginal pain by administering to a mammal in need of such treatment a therapeutically effective amount of a compound which acts upon mid-brain pathways to stimulate peripheral nerve release of nitric oxide (NO) in the pelvic nerve network. The vaginal pain may be general hyperalgesia (non-specific increased vaginal sensitivity) or pain associated with intercourse (dyspareunia).

The selected compound is one which acts upon any of the mid-brain pathways which include the dopaminergic, serotonergic, oxytocinergic or nitroxidergic mid-brain pathways.

In another embodiment, the present invention provides a method for producing an effective vasocongestive arousal in a female comprising administering a therapeutically effective amount of a compound which acts upon a mid-brain dopaminergic, serotonergic, oxytocinergic or nitroxidergic pathway to increase blood flow to the ilio-hypogastric-pudendal arterial bed. By effective vasocongestive arousal is meant clitoral erection, vaginal and labialar engorgement, and lubrication adequate for intercourse.

In yet another embodiment, the present invention provides a means of treating vaginal engorgement insufficiency in a female mammal comprising administering a therapeutically effective amount of a compound which acts upon a mid-brain dopaminergic, serotonergic, oxytocinergic or nitroxidergic pathway to increase blood flow to the ilio-hypogastric-pudendal arterial bed.

In another embodiment, the present invention provides a method of treating clitoral erectile insufficiency in a female mammal comprising administering a therapeutically effective amount of a compound which acts upon a mid-brain dopaminergic, serotonergic, oxytocinergic or nitroxidergic pathway to increase blood flow to the ilio-hypogastric-pudendal arterial bed.

In still another embodiment, the present invention comprises a method of treating dyspareunia in a female mammal comprising administering a therapeutically effective amount of a compound which acts upon a mid-brain dopaminergic, serotonergic, oxytocinergic or nitroxidergic pathway to facilitate peripheral nerve release of NO in the pelvic nerve network.

In the embodiments described above, an androgen may optionally be co-administered with the primary active compound, wherein co-administration of the androgen enhances or potentiates the effect of the principal therapeutic agent.

### BRIEF DESCRIPTION OF THE DRAWING

In the Drawing:

FIG. 1 is a histogram depicting yawning response of female test animals following administration of various doses of apomorphine.

FIG. 2 is a histogram depicting yawning response of female test animals at various times following the administration of apomorphine after pre-administration of testosterone.

### DETAILED DESCRIPTION

As used throughout this specification and the appended claims, the following terms have the meanings ascribed to them.

By “androgen” is meant testosterone, dihydro-testosterone, and dehydroepiandrostenedione, either in their free base forms or in the form of a salt or pro-drug.

The terms “acute dose” or “acute administration” of a drug means the scheduled administration of a drug to the patient on an as-needed basis.

The term “effective vasocongestive arousal” means, in the female, tumescent clitoral erection, engorgement, swelling and lubrication of the vagina and engorgement and swelling of the labia.

Compounds useful in the methods of the present invention are those compounds which are known to act upon the mesencephalon or mid-brain nerve pathways to increase blood flow to the ilio-hypogastric-pudendal arterial bed or to act on a mid-brain neural pathway to stimulate the peripheral release of nitric oxide (NO) from non-adrenergic, non-cholinergic (NANC) nerve cells in the pelvic region. These compounds include those which are known to act in any of the dopaminergic, serotonergic, oxytocinergic or nitroxidergic mammalian mid-brain pathways to produce such peripheral effects.

Dopaminergic pathway compounds include apomorphine, bromocriptine, lisuride, methergoline, pergolide, piribidil, and quinpirole.

Serotonergic pathway compounds include serotonin receptor agonists such as 1-(2,5-dimethoxy-4-iodophenyl)-laminopropane, 5-methoxytryptamine, a-methyl-5-hydroxytryptamine, 2-methyl-5-hydroxytryptamine, N-acetyl-5-hydroxytryptamine buspirone, and sumatriptin. Oxytocinergic pathway compounds include oxytocin analogues such as isotocin, carbetocin, Lys-conopressin, deaminooxytocin, mesotocin, antocin, glumitocin, aspargitocin, valitocin, asvatocin, phasvatocin, and seritocin.

The preferred compound for use in the methods of the present invention is apomorphine or one of its salts, esters or pro-drug forms. Apomorphine, (R)-5,6,6a,7-tetrahydro-6-methyl-(4H)-dibenzo [de,g]quinoline-10,11-diol, is a derivative of morphine obtained by treatment of the latter with concentrated hydrochloric acid (L. Small, et al., J. Org. Chem., 5: 334 (1940)) or by heating morphine with zinc chloride (Mayer, Ber., 4: 171 (1871)). The compound has the chemical structure:

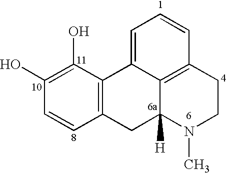

and possesses a chiral center at position 6a. The total synthesis of the racemic mixture is reported by J. L. Neumeyer, et al., J. Pharm. Sci., 59:1850 (1970) and the synthesis of the separate enantiomers by V. J. Ram and J. Neumeyer, J. Org. Chem ., 46: 2830 (1981).

The compound possesses a basic nitrogen atom at position 6 and is thus capable of existing in the free base form as well as acid addition salt forms. The compound may be administered as the free base or in the form of one of its pharmaceutically acceptable salts or pro-drug derivatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977). The salts are prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-ydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

The term “pro-drug” refers to compounds that are rapidly transformed in vivo to yield the parent compound, as for example, by hydrolysis in blood. T. Higuchi and V. Stella provide a thorough discussion of the pro-drug concept in “Pro-drugs as Novel Delivery Systems”, Vol. 14 of the A.C.S. Symposium Series, American Chemical Society (1975). Examples of esters useful as pro-drugs for compounds containing carboxyl groups may be found on pages 14-21 of “Bioreversible Carriers in Drug Design: Theory and Application,” edited by E. . Roche, Pergamon Press (1987).

The term “pro-drug ester group” refers to any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters includes formates, acetates, propionates, butryates, acrylates and ethylsuccinates.

Apomorphine has been shown to be effective in facilitating and maintaining erectile response in males. Formulations containing apomorphine for this purpose, and methods of treating erectile dysfunction in males is disclosed in U.S. Pat. (Ser. No. 08/546,498), the entire contents of which are incorporated herein by reference.

For an optimal vasocongestive arousal response in the female, steady state circulating serum and mid-brain tissue levels of apomorphine should be maintained within a relatively closely defined range. The drug is preferably administered in a formulation which rapidly delivers the drug to the system while maintaining and not exceeding the desired systemic levels of the drug. Methods known to the practitioner of the pharmaceutical formulation arts which accomplishes this means may be used. For example, the drug may be delivered to the system by means of a solid oral formulation, by a liquid formulation, including one applied sub-lingually; by a tablet, lozenge, or lollipop held in the mouth and absorbed buccally; by means of a suppository formulation administered intravaginally or rectally; by a powder, gel, or suspension, or an intra-nasal spray formulation.

The drug may also be administered in a sterile parenteral formulation by sub-cutaneous or intramuscular route, although sub-lingual, buccal, intra-nasal, and suppository formulations are preferred because of their greater ease of administration and the resulting greater potential for patient acceptance.

Sublingual apomorphine dosage forms, usually containing about 2.5 to about 10 milligrams of apomorphine, are useful in treating the symptoms of female vasculogenic sexual dysfunction, including its symptomatic manifestations without nausea or other undesirable side effects.

The apomorphine is administered in the time period immediately prior to sexual intercourse, generally during the period between about 2 minutes and 120 minutes prior to sexual relations, preferably during the period between about 2 minutes and about 60 minutes prior to sexual relations so as to maintain a predetermined circulating serum levels and mid-brain tissue levels of apomorphine during the period of sexual activity sufficient to induce clitoral erection, vaginal and labialar engorgement and lubrication adequate for intercourse (i.e. “effective vasocongestive arousal”) but less than the amount that induces nausea. Plasma concentrations of apomorphine are preferably maintained at between about 0.3 to 5.5 nanograms per milliliter, preferably between about 0.3 to about 4 nanograms per milliliter, and more preferably between about 1 to about 2 nanograms per milliliter.

Apomorphine is a dopamine receptor agonist that has a recognized use as an emetic when administered subcutaneously in about a 5-milligram dose. For the purposes of the present invention, apomorphine or a similarly acting dopamine receptor agonist is administered in an amount sufficient to excite cells in the mid-brain region of the patient but with minimal side effects. This cell excitation is believed to be part of a cascade of stimulation that is likely to include neurotransmission with serotonin and oxytocin.

The dopamine receptors in the mid-brain region of a patient can be stimulated to a degree sufficient to cause an erectile response by the administration, preferably sublingually, of apomorphine so as to maintain a plasma concentration of apomorphine of no more than about 5.5 nanograms per milliliter (5.5 ng/ml). The sublingual administration usually takes place over a time period in the range of about 2 to about 10 minutes, or longer. The amount of apomorphine administered sublingually over this time period preferably is in the range of about 25 micrograms per kilogram (μg/kg) of body weight to about 60 μg/kg of body weight.

Illustrative preferred sublingual dosage forms are set forth in Table I, below.

TABLE I
150-Milligram Apomorphine Hydrochloride
Sublingual Tablets
3-mg Tablet

| | | |
|---|---|---|
| Apomorphine Hydrochloride | 2.00 | wt-% |
| Mannitol | 66.67 | wt-% |
| Ascorbic Acid | 3.33 | wt-% |
| Citric Acid | 2.00 | wt-% |
| Avicel PH102 | 15.00 | Wt-% |
| Methocel E4 | 10.00 | Wt-% |
| Aspartame | 0.67 | wt-% |
| Magnesium stearate | 0.33 | wt-% |

4-mg Tablet

| | | |
|---|---|---|
| Apomorphine Hydrochloride | 2.66 | wt-% |
| Mannitol | 66.00 | wt-% |
| Ascorbic Acid | 3.33 | wt-% |
| Citric Acid | 2.00 | wt-% |
| Avicel PH102 | 15.00 | wt-% |
| Methocel E4M | 10.00 | Wt-% |
| Aspartame | 0.67 | wt-% |
| Magnesium stearate | 0.33 | wt-% |

5-mg Tablet

| | | |
|---|---|---|
| Apomorphine Hydrochloride | 3.33 | wt-% |
| Mannitol | 65.34 | wt-% |
| Ascorbic Acid | 3.33 | wt-% |
| Citric Acid | 2.00 | wt-% |
| Avicel PH102 | 15.00 | wt-% |
| Methocel E4M | 10.00 | wt-% |
| Aspartame | 0.67 | wt-% |
| Magnesium stearate | 0.33 | wt-% |

If desired, and in order to facilitate absorption and thus bioavailability, the presently contemplated dosage forms can also contain, in addition to tabletting excipients, β-cyclodextrin or a β-cyclodextrin derivative such as hydroxypropyl-β-cyclodextrin (HPBCD). Illustrative dosage forms containing HPBCD are shown in Tables II and III, below.

TABLE II
Apomorphine Hydrochloride Sublingual Tablets
With Hydroxypropyl-β-Cyclodextrin

| | mg/Tab |
|---|---|
| Apomorphine hydrochloride | 4.0 |
| HPBCD | 5.0 |
| Ascorbic acid | 10.0 |
| PEG 8000 | 39.5 |
| Mannitol | 39.5 |
| Aspartame | 2.0 |
| Total | 100.0 |

TABLE III
Apomorphine Hydrochloride Sublingual
Tablets With β-Cyclodextrin

| | mg/Tab |
|---|---|
| Apomorphine hydrochloride | 5.0 |
| β-Cyclodextrin | 20.0 |
| Ascorbic acid | 5.0 |
| Mannitol | 68.9 |
| Magnesium stearate | 1.0 |
| D&C Yellow 10 aluminum lake | 0.1 |
| TOTAL | 100.0 |

The onset of nausea can be obviated or delayed by delivering apomorphine at a controlled dissolution rate so as to provide circulating serum levels and midbrain tissue levels of apomorphine sufficient for an effective vasoconstrive arousal without inducing nausea. When apomorphine is administered at or near the relatively higher amounts of the aforementioned dosage range, the likelihood of nausea onset can be reduced by concurrent administration of a ganglionic agent (inhibitor of ganglionic response) such as nicotine or lobeline sulfate. For this purpose, the weight ratio of apomorphine to ganglionic agent is in the range of about 10 to about 1.

Other antiemetic agents that can be used in conjunction with apomorphine are antidopaminergic agents such as metoclopramide, and the phenothiazines, e.g., chlorpromazine, prochlorperazine, pipamazine, thiethylperazine, oxypendyl hydrochloride, and the like. Also suitable are the serotonin (5-hydroxytryptamine or 5-HT) antagonists such as domperidone, ondansetron (commercially available as the hydrochloride salt under the designation Zofran©), and the like, the histamine antagonists such as buclizine hydrochloride, cyclizine hydrochloride, dimenhydrinate (Dramamine), and the like, the parasympathetic depressants such as scopolamine, and the like, as well as other anti-emetics such as metopimazine, trimethobenzamide, benzauinamine hydrochloride, diphenidol hydrochloride, and the like.

Nicotine-containing dosage forms and domperidone-containing dosage forms are illustrated in Table IV, below.

TABLE IV
Apomorphine Hydrochloride Sublingual Tablets
Containing an Anti-Emetic Agent

| | mg/Tab |
|---|---|
| Apomorphine Hydrochloride | 5.0 |
| Ascorbic Acid | 5.0 |
| Mannitol | 67.9 |
| Magnesium Stearate | 1.0 |
| Nicotine | 1.0 |
| β-Cyclodextrin | 20.0 |
| D&C Yellow aluminum lake | 0.1 |
| TOTAL | 100.0 |
| Apomorphine Hydrochloride | 5.0 |
| Ascorbic Acid | 5.0 |
| Mannitol | 58.9 |
| Magnesium Stearate | 1.0 |
| Domperidone | 10.0 |
| β-Cyclodextrin | 20.0 |
| D&C Yellow 10 aluminum lake | 0.1 |
| TOTAL | 100.0 |

The preferred sublingual dosage forms dissolve within a time period of at least about 2 minutes but less than about 10 minutes. The dissolution time can be longer, however, if desired as long as the necessary plasma concentration of apomorphine can be maintained. More preferably, the dissolution time in water for the presently contemplated dosage forms is about 3 minutes to about 5 minutes.

The present invention is illustrated further by the following studies. Eight Wistar rats were acquired with 250-275 gm body weight and were housed in individual cages with food and water ad libitum. Animals were placed separately in hanging test cages with transparent plastic bottoms located in a dark, soundproof room and allowed to aclimate for tenb minutes. A solution of apomorphine hydrochloride, 120 micrograms/mL, with 100 micrograms/mL of ascorbic acid in physiological saline was prepared and administered subcutaneously to the back of the neck of the test animals. Randomized blind testing was performed with doses of 40 micrograms/kg, 80 micrograms/kg, and 120 micrograms/kg, using vehicle for control. Doses were obtained by administering different amounts of the stock solution of 120 micrograms/mL.

In a separate series of tests, testosterone was administered at a dosage of 480 micrograms/kg, with doses of apomorphine being subsequently administered at times 0, 24, 36 and 48 following administration of the testosterone.

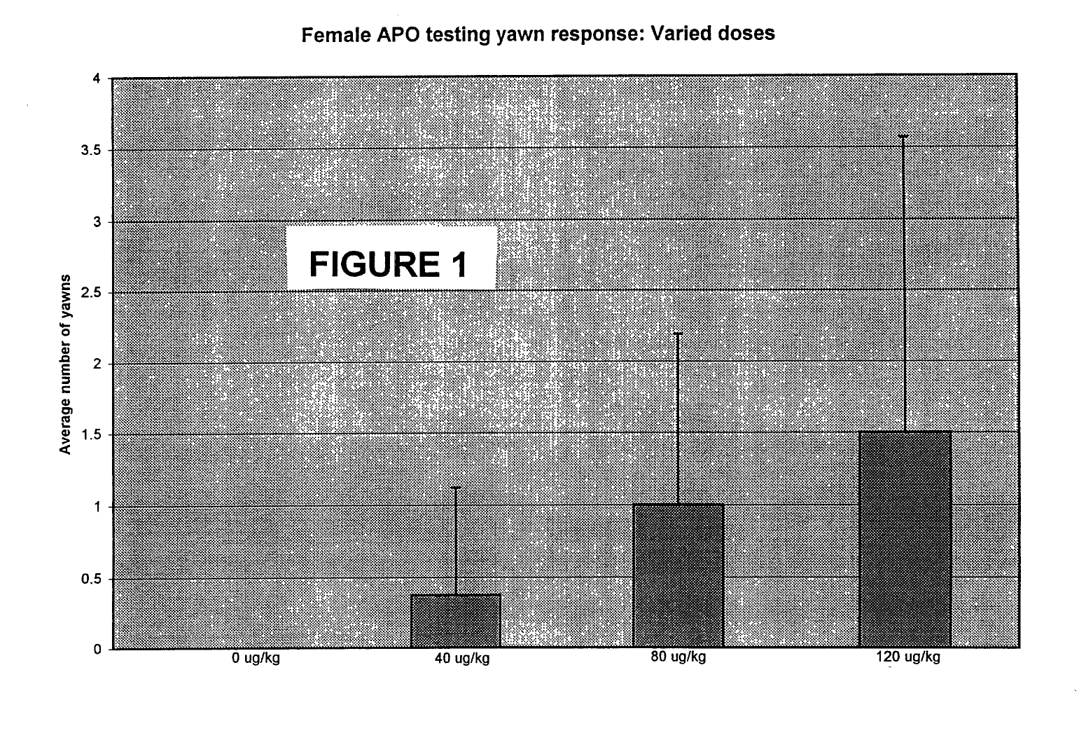

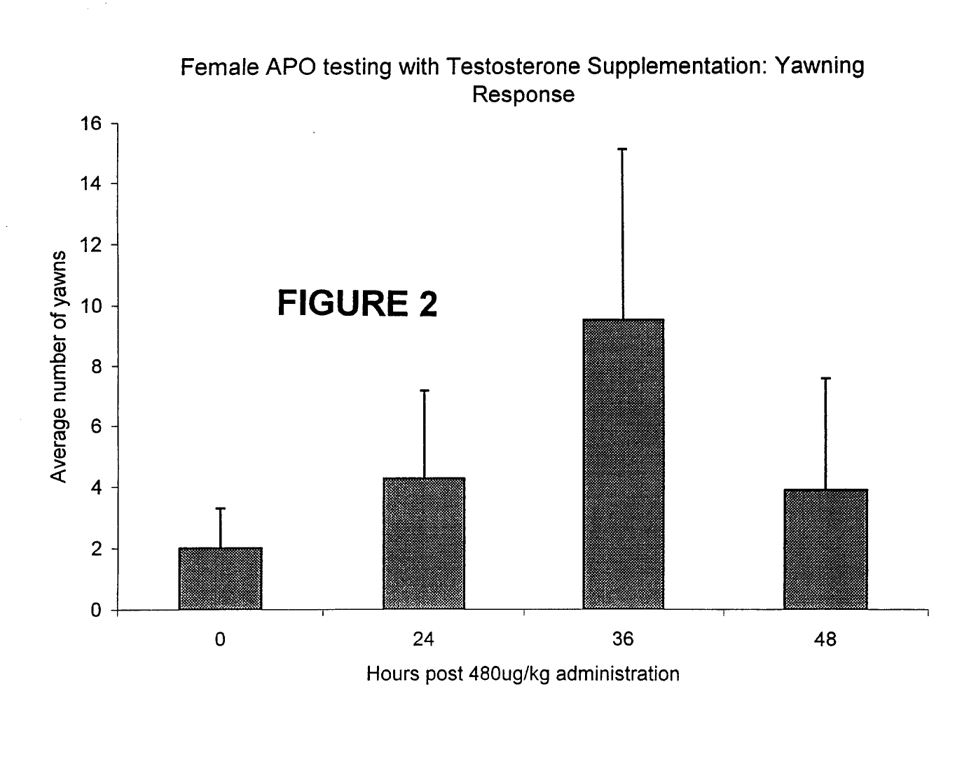

Following administration of the drug, the animals were observed for yawning behaviour, a response which accompanies and is indicative of sexual arousal, and for hunching and licking of the genital area which behaviors evince increased awareness of the genital area. The results are presented in FIGS. 1 and 2 where FIG. 1 shows a dose-dependent yawning response in the test animals. In FIG. 2 where testosterone was pre-administered to the test animals, a maximum yawing response was observed 36 hours following the initial testosterone administration.

The foregoing discussion and the reported studies are intended as illustrative of the present invention and are not to be read as limiting the invention as it is defined by the appended claims.

We claim:

1. A method of treating vasculogenic sexual dysfunction in a female mammal in need of such treatment which comprises administering to said female a therapeutically effective amount of a compound which acts upon a mid-brain pathway to increase blood flow in the ilio-hypogastric-pudendal arterial bed.

2. A method according to claim 1 wherein said compound acts upon a dopaminergic mid-brain pathway to increase blood flow in the ilio-hypogastric-pudendal arterial bed.

3. A method according to claim 1 wherein said compound acts upon a serotonergic mid-brain pathway to increase blood flow in the ilio-hypogastric-pudendal arterial bed.

4. A method according to claim 1 wherein said compound acts upon an oxytocinergic mid-brain pathway to increase blood flow in the ilio-hypogastric-pudendal arterial bed.

5. A method according to claim 1 wherein said compound acts upon a nitroxidergic mid-brain pathway to increase blood flow in the ilio-hypogastric-pudendal arterial bed.

6. A method according to claim 2 wherein said compound acting upon a mid-brain dopaminergic pathway is selected from the group consisting of apomorphine, bromocriptine, lisuride, methergoline, pergolide, pribidil and quinapril or a pharmaceutically acceptable salt, ester, or pro-drug thereof.

7. A method according to claim 3 wherein said compound acting upon a mid-brain serotonergic pathway is selected from the group consisting of 1-(2,5-dimethoxy-4-iodophenyl)-1-aminopropane, 5-methoxytryptamine, α-methyl-5-hydroxytryptamine, 2-methyl-5-hydroxytryptamine, N-acetyl-5-hydroxytryptamine, buspirone, and sumatriptin or a pharmaceutically acceptable salt, ester, or pro-drug thereof.

8. A method according to claim 4 wherein said compound acting upon a mid-brain oxytocinergic pathway is selected from the group consisting of isotocin, carbetocin, Lys-conopressin, deaminooxytocin, mesotocin, antocin, glumitocin, aspargitocin, valitocin, asvatocin, phasvatocin, and seritocin or a pharmaceutically acceptable salt, ester, or pro-drug thereof.

9. A method of treating vasculogenic sexual dysfunction in a female in need of such treatment which comprises administering to a patient in need of such treatment a therapeutically effective amount of apomorphine of a pharmaceutically acceptable salt, ester, or pro-drug thereof.

10. The method of claim 9 wherein said apomorphine is administered in an amount sufficient to produce an effective vasocongestive arousal in said female, but insufficient to induce nausea.

11. The method of claim 10 wherein said apomorphine is administered in an amount between about 25 micrograms/kg of body weight and about 60 micrograms/kg of body weight.

12. The method of claim 9 wherein said apomorphine is administered in an amount sufficient to establish plasma concentration levels of apomorphine ranging between about 0.3 to about 5.5 nanograms/mL.

13. The method of claim 12 wherein said plasma levels of apomorphine range between about 0.3 and about 4 nanograms/mL.

14. The method of claim 13 wherein said plasma levels of apomorphine range between about 1 and about 2 nanograms/mL.

15. A method of inducing effective vasocon-strictive arousal in a female in need of such treatment comprising administering a therapeutically effective amount of apomorphine or a pharmaceutically effective salt, ester, or pro-drug thereof.

16. The method of claim 15 wherein said apomorphine is administered in the interval between about 120 minuites and 2 minutes prior to coitus.

17. The method of claim 16 wherein said apomorphine is administered over a period of time ranging between about two to about ten minutes.

18. The method of claim 17 wherein said apomorphine is administered sublingually.

19. A method of treating vaginal engorgement insufficiency in a female in need of such treatment comprising administering a therapeutically effective amount of apomorphine or a pharmaceutically acceptable salt, ester or pro-drug thereof.

20. The method of claim 19 wherein said apomorphine is administered in an amount between about 25 micrograms/kg of body weight and about 60 micrograms/kg of body weight.

21. The method of claim 20 wherein said apomorphine is administered in an amount sufficient to establish plasma concentration levels of apomorphine ranging between about 0.3 to about 5.5 nanograms/mL.

22. The method of claim 21 wherein said plasma levels of apomorphine range between about 0.3 and about 4 nanograms/mL.

23. The method of claim 22 wherein said plasma levels of apomorphine range between about 1 and about 2 nanograms/mL.

24. The method of claim 19 wherein said apomorphine is administered in the interval between about 120 minuites and 2 minutes prior to coitus.

25. The method of claim 24 wherein said apomorphine is administered over a period of time ranging between about two to about ten minutes.

26. The method of claim 19 wherein said apomorphine is administered sublingually.

27. A method of treating clitoral erectile insufficiency in a female in need of such treatment comprising administering a therapeutically effective amount of apomorphine or a pharmaceutically acceptable salt, ester or pro-drug thereof.

28. The method of claim 27 wherein said apomorphine is administered in an amount between about 25 micrograms/kg of body weight and about 60 micrograms/kg of body weight.

29. The method of claim 27 wherein said apomorphine is administered in an amount sufficient to establish plasma concentration levels of apomorphine ranging between about 0.3 to about 5.5 nanograms/mL.

30. The method of claim 29 wherein said plasma levels of apomorphine range between about 0.3 and about 4 nanograms/mL.

31. The method of claim 30 wherein said plasma levels of apomorphine range between about 1 and about 2 nanograms/mL.

32. The method of claim 27 wherein said apomorphine is administered in the interval between about 120 minutes and 2 minutes prior to coitus.

33. The method of claim 32 wherein said apomorphine is administered over a period of time ranging between about two to about ten minutes.

34. The method of claim 32 wherein said apomorphine is administered sublingually.

35. A method of combating vaginal pain comprising administering to a female in need of such treatment a compound which acts upon mid-brain neural pathways to stimulate peripheral pelvic non-adrenergic non-cholinergic (NANC) nerve cell release of nitric oxide (NO).

36. A method according to claim 35 wherein said compound acts upon a dopaminergic mid-brain pathway to stimulate peripheral pelvic NANC cell release of nitric oxide (NO).

37. A method according to claim 35 wherein said compound acts upon a serotonergic mid-brain pathway to stimulate peripheral pelvic NANC nerve cell release of nitric oxide (NO).

38. A method according to claim 35 wherein said compound acts upon an oxytocinergic mid-brain pathway to stimulate peripheral pelvic NANC nerve cell release of nitric oxide (NO).

39. A method according to claim 35 wherein said compound acts upon a nitroxidergic mid-brain pathway to stimulate peripheral pelvic NANC nerve cell release of nitric oxide (NO).

40. A method according to claim 36 wherein said compound acting upon a mid-brain dopaminergic pathway is selected from the group consisting of apomorphine, bromocriptine, lisuride, methergoline, pergolide, pribidil and quinapril or a pharmaceutically acceptable salt, ester, or pro-drug thereof.

41. A method according to claim 37 wherein said compound acting upon a mid-brain serotonergic pathway is selected from the group consisting of 1-(2,5-dimethoxy-4-iodophenyl)-1-aminopropane, 5-methoxytryptamine, α-methyl-5-hydroxytryptamine, 2-methyl-5-hydroxytryptamine, N-acetyl-5-hydroxytryptamine, buspirone, and sumatriptin or a pharmaceutically acceptable salt, ester, or pro-drug thereof.

42. A method according to claim 39 wherein said compound acting upon a mid-brain oxytocinergic pathway is selected from the group consisting of isotocin, carbetocin, Lys-conopressin, deaminooxytocin, mesotocin, antocin, glumitocin, aspargitocin, valitocin, asvatocin, phasvatocin, and seritocin or a pharmaceutically acceptable salt, ester, or pro-drug thereof.

43. A method of treating vaginal pain in a female in need of such treatment which comprises administering to a patient in need of such treatment a therapeutically effective amount of apomorphine of a pharmaceutically acceptable salt, ester, or pro-drug thereof.

44. The method of claim 43 wherein said apomorphine is administered in an amount between about 25 micrograms/kg of body weight and about 60 micrograms/kg of body weight.

45. The method of claim 42 wherein said apomorphine is administered in an amount sufficient to establish plasma concentration levels of apomorphine ranging between about 0.3 to about 5.5 nanograms/mL.

46. The method of claim 45 wherein said plasma levels of apomorphine range between about 0.3 and about 4 nanograms/mL.

47. The method of claim 46 wherein said plasma levels of apomorphine range between about 1 and about 2 nanograms/mL.

48. The method of claim 35 wherein said apomorphine is administered sublingually.

49. A method of treating dyspareunia in a female in need of such treatment comprising administering a therapeutically effective amount of apomorphine or a pharmaceutically acceptable salt, ester, or pro-drug thereof.

50. The method of claim 49 wherein said apomorphine is administered in an amount between about 25 micrograms/kg of body weight and about 60 micrograms/kg of body weight.

51. The method of claim 49 wherein said apomorphine is administered in an amount sufficient to establish plasma concentration levels of apomorphine ranging between about 0.3 to about 5.5 nanograms/mL.

52. The method of claim 51 wherein said plasma levels of apomorphine range between about 0.3 and about 4 nanograms/mL.

53. The method of claim 52 wherein said plasma levels of apomorphine range between about 1 and about 2 nanograms/mL.

54. The method of claim 49 wherein said apomorphine is administered in the interval between about 120 minutes and 2 minutes prior to coitus.

55. The method of claim 49 wherein said apomorphine is administered over a period of time ranging between about two to about ten minutes.

56. The method of claim 41 wherein said apomorphine is administered sublingually.
